(19) United States
(12) Patent Application Publication
(10) Pub. No.: US 2020/0388390 A1
(43) Pub. Date: Dec. 10, 2020

(54) DIGITAL HEALTH PROXY SYSTEM AND DEVICE

(71) Applicant: KONINKLIJKE PHILIPS N.V., EINDHOVEN (NL)

(72) Inventors: Portia E. SINGH, Everett, MA (US); Saman PARVANEH, Danvers, MA (US)

(21) Appl. No.: 16/772,534

(22) PCT Filed: Dec. 21, 2018
(86) PCT No.: PCT/EP2018/086478

§ 371 Date: Jun. 12, 2020

Related U.S. Application Data
(60) Provisional application No. 62/609,911, filed on Dec. 22, 2017.

(51) Int. Cl. G16H 50/20; G16H 10/60; G16H 70/20; G16H 50/70; G16H 10/20; G16H 20/10; G06Q 40/08; G06F 16/9535; H04L 29/06; H04N 7/18; G06F 40/40

(52) U.S. Cl. CPC G16H 50/20; G16H 10/60; G16H 70/20; G16H 50/70; G16H 10/20; G16H 20/10; H04L 67/26; G06F 16/9535; H04L 63/20; H04L 63/08; H04N 7/183; G06F 40/40; G06Q 40/08

(57) ABSTRACT

The present invention relates to a digital health proxy device, system, and method. or particularly, but not exclusively, the various apparatuses, methods, and systems disclosed herein relate to a health proxy device configured to parse medical data using data processing techniques and provide assistive technologies to a caregiver and/or patient for making an optimal medical decision in a time-sensitive environment.

### FIELD OF THE DISCLOSURE

The present invention relates to a digital health proxy device, system, and method. or particularly, but not exclusively, the various apparatuses, methods, and systems disclosed herein relate to a health proxy device configured to parse medical data using data processing techniques and provide assistive technologies to a caregiver and/or patient for making an optimal medical decision in a time-sensitive environment.

### BACKGROUND OF THE DISCLOSURE

For informal caregivers (ICG), accompanying their loved one (e.g., a child or an elder loved one) to the emergency department during an acute event can be a stressful, frightening and overwhelming time. The informal caregiver may need to be the voice of the patient even if he or she does not understand the situation fully themselves. Because of this, informal caregivers rely heavily on the direction of the physician and the physician recommendations, which may or may not be the correct course of action for the care recipient.

To the extent a caregiver relies upon additional resources, those resources may simply be generic websites such as medical and/or health news and publications (e.g., WebMD®). However, parsing through online and/or printed materials is time-consuming and ineffective in time-sensitive scenarios such as the acute care setting. Additionally, the information may not be available in a manner that is easily understood by a lay person and therefor is an ineffective resource for many individuals. Accordingly, by relying solely on a medical professional, a caregiver may not be thoroughly aware of alternative testing, diagnosis or treatment options. And, as a result, a caregiver may not be able to identify the best testing, diagnosis, and/or treatment options specific to the care recipient based at least on the care recipient's specific medical history.

In addition, current systems and devices lack the ability to provide medically secure assistance and optimized assistive technology. For example, a device that may operate the assistive technology may include a caregiver device that may at times remain unsecured (e.g., unlocked, open to public wireless networks, and/or the like). It is estimated that about 250,000 people die in the United States alone due to medical errors every year. Given an advanced medical proxy device with at least enhanced medical databasing and an improved processing technologies as described herein, these drawbacks in current technologies may be addressed so that caregivers may gather diagnosis, treatment, protocol, and clinical data and/or determine an optimal treatment plan for the care recipient.

### SUMMARY OF THE DISCLOSURE

The present disclosure is directed to inventive methods and apparatus for a device, system, and methods that provide health proxy technologies. Generally, in one aspect a caregiver device is disclosed where the caregiver device includes an antenna that communicates wirelessly with a network; an input/output interface comprising a display; and a processor that: receives an indication to begin a medical guidance session; transmits, via the antenna and in response to receiving an indication to begin a medical guidance session, a request to generate a dynamic database comprising patient specific data, population-based clinical data, and medical ontology data, wherein the dynamic database is dynamically reduced in response to each query to the dynamic database during the medical guidance session; receives current patient event data comprising a treatment recommendation and at least one of: diagnosis data, clinical notes, and image data; executes a query process comprising: querying the dynamic database using the current patient event data to identify a refined dataset used to determine (a) a preliminary treatment confidence associated with the treatment recommendation and (b) additional data that could increase the preliminary treatment confidence; generating a question for the user, wherein the question is associated with the determined additional data; receiving a response to the generated question via the input/output interface; and determining an updated treatment confidence associated with the treatment recommendation; and transmits, via the input/output interface, caregiver data comprising at least one of: the updated treatment confidence and a description of the treatment recommendation associated with the medical guidance session.

In some aspects of the invention the processor further determines a security setting based on network settings associated with the network, and wherein the indication to begin a medical guidance session comprises a security feature based on the security setting comprising at least one of: a username, a password, a personal identification number (PIN), and a biometric data.

In some aspects of the invention the processor, prior to receiving the indication to begin the medical guidance session, transmits a notification to change a network setting to increase security.

Generally, in some aspects of the invention the patient specific data comprises at least one of: prior medical record data, caregiver observation data, personal emergency response system (PERS) data, care recipient wearable device data, medication adherence data, and medication data.

Generally, in some aspects of the invention the query process is repeated until the updated treatment confidence is above a predetermined threshold, wherein for each repetition, the preliminary treatment confidence is substituted with a further updated treatment confidence. In still other aspects of the invention, wherein the treatment recommendation is changed when the updated treatment confidence associated with the treatment recommendation drops below a predetermined threshold.

Generally, in some aspects of the invention the patient specific data comprises patient insurance data, and wherein at least one of the preliminary treatment confidence and updated treatment confidence is based on the patient insurance data, and wherein the generated question is related to the patient insurance data.

In some aspects of the inventions, a device may further comprise a location detection unit that detects a location of the device, wherein when the location of the device is within a predefined distance of a medical facility, the processor generates a notification to begin the medical guidance session prior to receiving an indication to begin the medical guidance session. In still other aspects of the invention, when the location detection unit determines the location of the device is within a predefined distance of a medical facility, the device generates a notification to alter a network setting, wherein the notification comprises selectable data to automatically alter the network setting.

Generally, in some aspects of the invention, a device may further comprise a camera, wherein image data is received via the camera. In still other aspects of the invention, a treatment recommendation may comprise at least two treatment options, and wherein the preliminary treatment confidence comprises a treatment option confidence for each treatment option, and wherein the determined additional data could increase the treatment option confidence for at least one of the treatment options.

In some aspects, a device may further comprise a memory whereby the dynamic database is cached in the memory. Generally, in some aspects of the invention the dynamic database may be cached upon reaching a predefined size. In still some aspects of the invention, the antenna may become inactive once the dynamic database is cached on the device. And, in some aspects of the invention, the antenna may be and/or remain inactive until the medical guidance session ends.

In other aspects of the invention, an input/output interface of a device may further comprise a microphone, wherein at least a portion of the current patient event data is received via the microphone.

In other aspects of the invention, the current patient event data is preprocessed using natural language processing. In still other aspects, caregiver data may be preprocessed prior to transmission, wherein the preprocessing of the caregiver data comprises performing natural language processing on at least one of the updated treatment confidence and the description of the treatment recommendation. In other aspects of the invention, the caregiver data may further comprise at least one follow-up question.

Generally, in some aspects of the invention, determining the preliminary treatment confidence and the updated treatment confidence may comprise using a machine learning technique such as at least one of: decision tree learning, association rule learning, neural networks, deep learning, inductive logic programming (ILP), a support vector machine (SVM), clustering, Bayesian networks, reinforcement learning, and a learning classifier system (LCS). In still some aspects of the invention the preliminary treatment confidence and the updated treatment confidence are each based on matched cases.

### BRIEF DESCRIPTION OF THE DRAWINGS

Various embodiments of the present disclosure, together with further objects and advantages, may best be understood by reference to the following description taken in conjunction with the accompanying drawings, in the several Figures of which like reference numerals identify like elements, and in which:

FIG. 1 depicts an example embodiment of a system for providing health care proxy assistance according to embodiments of the disclosure;

FIG. 2 depicts an exemplary embodiment of a system for providing health care proxy assistance according to embodiments of the disclosure;

FIG. 3 depicts an exemplary embodiment of an apparatus used in a health care proxy assistance system according to embodiments of the disclosure; and

FIG. 4 depicts an exemplary embodiment of a method for providing health care proxy assistance according to embodiments of the disclosure.

### DETAILED DESCRIPTION OF THE EMBODIMENTS

The following description is intended to convey a thorough understanding of the embodiments described by providing a number of specific exemplary embodiments and details involving a digital health proxy device, system, and method thereof. It should be appreciated, however, that the present disclosure is not limited to these specific embodiments and details, which are examples only. It is further understood that one possessing ordinary skill in the art, in light of known systems and methods, would appreciate the use of the invention for its intended purposes and benefits in any number of alternative embodiments, depending on specific design and other needs.

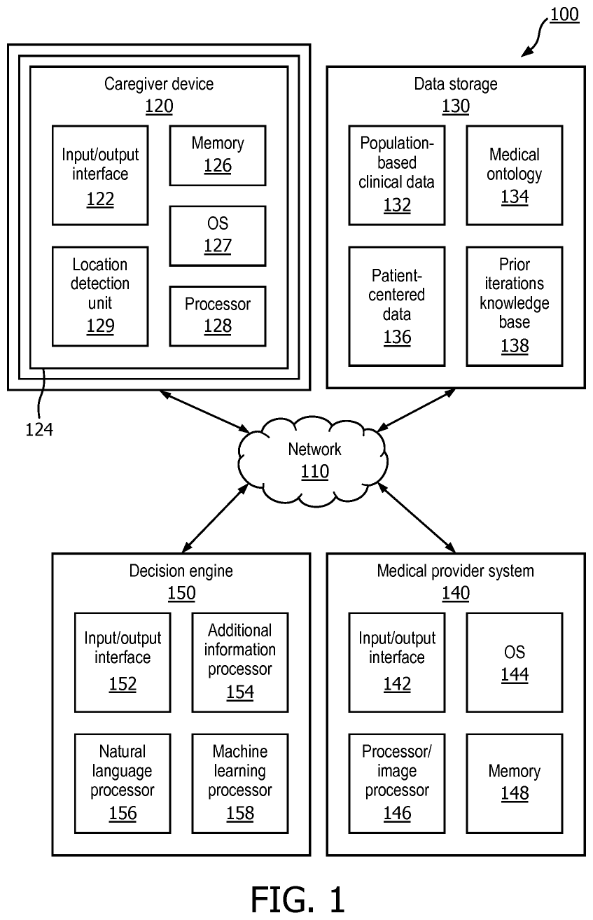

FIG. 1 depicts an exemplary system 100 that provides digital health proxy assistance. As shown in FIG. 1, an example system 100 may include a caregiver device 120, data storage 130, a medical provider system 140, and/or a decision engine 150. As described herein, the technical features of data storage 130 and/or decision engine 150 may be separate from caregiver device 120 and/or medical provider system 140 or may be combined with a caregiver device 120 and/or a medical provider system 140. Caregiver device 120, data storage 130, medical provider system 140, and decision engine 150 may all be connected via a network 110.

For example, network 110 may be one or more of a wireless network, a wired network or any combination of wireless network and wired network. For example, network 110 may include one or more of a fiber optics network, a passive optical network, a cable network, an Internet network, a satellite network, a wireless LAN, a Global System for Mobile Communication (“GSM”), a Personal Communication Service (“PCS”), a Personal Area Network (“PAN”), Wireless Application Protocol (WAP), Multimedia Messaging Service (MMS), Enhanced Messaging Service (EMS), Short Message Service (SMS), Time Division Multiplexing (TDM) based systems, Code Division Multiple Access (CDMA) based systems, D-AMPS, Wi-Fi, Fixed Wireless Data, IEEE 802.11b, 802.15.1, 802.11n and 802.11g, a Bluetooth network, or any other wired or wireless network for transmitting and receiving a data signal.

In addition, network 110 may include, without limitation, telephone lines, fiber optics, IEEE Ethernet 902.3, a wide area network (“WAN”), a local area network (“LAN”), a wireless personal area network (“WPAN”), or a global network such as the Internet. Also network 110 may support an Internet network, a wireless communication network, a cellular network, or the like, or any combination thereof. Network 110 may further include one network, or any number of the example types of networks mentioned above, operating as a stand-alone network or in cooperation with each other. Network 110 may utilize one or more protocols of one or more network elements to which they are communicatively coupled. Network 110 may translate to or from other protocols to one or more protocols of network devices. Although network 110 is depicted as a single network, it should be appreciated that according to one or more embodiments, network 110 may comprise a plurality of interconnected networks, such as, for example, the Internet, a service provider's network, a cable television network, corporate networks, and/or home networks.

Caregiver device 120 and/or medical provider system 140 may include, for example, one or more mobile devices, such as, for example, personal digital assistants (PDA), tablet computers and/or electronic readers (e.g., iPad, Kindle Fire, Playbook, Touchpad, etc.), wearable devices (e.g., Google Glass, Apple Watch, wearable medical devices, etc.), telephony devices, smartphones, cameras, music playing devices (e.g., iPod, etc.), televisions, set-top-box devices, and the like.

Caregiver device 120, data storage 130, medical provider system 140, and/or decision engine 150 may include a network-enabled computer system and/or device. As referred to herein, a network-enabled computer system and/or device may include, but is not limited to: e.g., any computer device, or communications device including, e.g., a server, a network appliance, a personal computer (PC), a workstation, a mobile device, a phone, a handheld PC, a personal digital assistant (PDA), a thin client, a fat client, an Internet browser, or other device. The network-enabled computer systems may execute one or more software applications to, for example, receive data as input from an entity accessing the network-enabled computer system, process received data, transmit data over a network, and receive data over a network.

Caregiver device 120, data storage 130, medical provider system 140, and/or decision engine 150 may include at least one central processing unit (CPU), which may be configured to execute computer program instructions to perform various processes and methods. Caregiver device 120, data storage 130, medical provider system 140, and/or decision engine 150 may include data storage, including for example, random access memory (RAM) and read only memory (ROM), which may be configured to access and store data and information and computer program instructions. Data storage may also include storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium), where the files that comprise an operating system, application programs including, for example, web browser application, email application and/or other applications, and data files may be stored. The data storage of the network-enabled computer systems may include electronic information, files, and documents stored in various ways, including, for example, a flat file, indexed file, hierarchical database, relational database, such as a database created and maintained with software from, for example, Oracle® Corporation, Microsoft® Excel file, Microsoft® Access file, or any other storage mechanism.

Caregiver device 120, data storage 130, medical provider system 140, and/or decision engine 150 may further include, for example, a processor, which may be several processors, a single processor, or a single device having multiple processors. Although depicted as single elements, it should be appreciated that according to one or more embodiments, caregiver device 120, data storage 130, medical provider system 140, and/or decision engine 150 may comprise a plurality of caregiver devices 120, data storage 130, medical provider systems 140, and/or decision engines 150.

As depicted in FIG. 1, system 100 may include caregiver device 120. Caregiver device may include an input/output interface 122, antenna 124, memory 126, an operating system 127, a processor 128, and/or a location detection unit 129. Input/output interface 122 may include for example, I/O devices, which may be configured to provide input and/or output to/from caregiver device 120 (e.g., keyboard, mouse, touch input, camera, microphone, display, speakers, printers, modems, network cards, etc.). Input/output interface also may include antennae interface(s) and/or network interface(s) that may provide or enable wireless and/or wire line digital and/or analog interface to one or more networks, such as network 110, over one or more network connections, a power source that provides an appropriate alternating current (AC) or direct current (DC) to power one or more components of caregiver device 120, and a bus that allows communication among the various components of caregiver device 120. Input/output interface 122 may include a display, which may include for example output devices, such as a printer, display screen (e.g., monitor, television, and the like), speakers, projector, and the like. Although not shown, caregiver device 120 may include one or more encoders and/or decoders, one or more interleavers, one or more circular buffers, one or more multiplexers and/or de-multiplexers, one or more permuters and/or depermuters, one or more encryption and/or decryption units, one or more modulation and/or demodulation units, one or more arithmetic logic units and/or their constituent parts, and the like.

Input/output interface 122 may be capable of utilizing standardized transmission protocols, for example but not by way of limitation, ISO/IEC 14443 A/B, ISO/IEC 18092, MiFare, FeliCa, tag/smartcard emulation, and the like. Also, input/output interface 122 may be able to utilize transmission protocols and methods that are developed in the future using other frequencies or modes of transmission. Input/output interface 122 may also be backwards-compatible with existing techniques, for example RFID. Also, input/output interface 122 may support transmission requirements to meet new and evolving standards including internet based transmission triggered by NFC.

Antennae 124 may include any antenna configured to enable wireless communications such as Bluetooth, Bluetooth Low Energy (BLE), radio frequency identification (RFID), near field communication (NFC), WiFi, and/or the like.

Memory 126 may include various types of data storage. By way of example, memory 126 may include random access memory (RAM) and read only memory (ROM), which may be configured to access and store data and information and computer program instructions. Memory 126 may include cached data storage (e.g., CPU cache, GPU cache, digital signal processors, memory management unit, disk cache, web cache, and/or the like). Memory 126 may also include storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium), where the files that comprise an operating system, application programs including, for example, web browser application, email application and/or other applications, and data files may be stored. Memory 126 may include electronic information, files, and documents stored in various ways, including, for example, a flat file, indexed file, hierarchical database, relational database, such as a database created and maintained with software from, for example, Oracle® Corporation, Microsoft® Excel file, Microsoft® Access file, or any other storage mechanism.

Operating system 127 may store instructions that operate various hardware components of the caregiver device such as input/output interface 122, antenna 124, memory 126, processor 128, and/or location detection unit 129. By way of example, operating system may store various setting parameters such as network setting parameters, location detection parameters and/or input/output parameters. Operating system may also store settings associate with various applications running on a caregiver device 120, such as a health proxy assistance application as described herein. Applications, such as a health proxy assistance application may have instructions stored in memory, such as memory 126.

Processor 128 may include hardware and/or software required to execute operating systems software and/or various health proxy assistance methods described herein. For example, processor 128 may accept input data via the input/output interface 122 and may perform data alignment or cleaning and/or natural language processing in order to best interpret the received data in real time.

Location detection unit 129 may include hardware and/or software components capable of detecting the location of caregiver device 120. By way of example, location detection unit 129 may receive data via antenna 124 associated with the location of the caregiver device. Location detection unit may determine a position of a caregiver device (e.g., longitudinal/latitudinal, other coordinate, address-level, and/or otherwise); may determine the position of a caregiver device in relation to another location (e.g., a medical facility such as a doctor's office, a hospital, a clinic, and/or the like) by receiving proximity data associated with RFID, BLE, Bluetooth, WiFi, NFC, and/or other network data linked to a specific location (e.g., a BLE transmission of a universally unique identifier, UUID, wherein the UUID is associated with a known location and/or device); GPS and/or coordinate data similar to and/or associated with a known location and/or a known location type; and/or the like. Proximity data may further include, for example, an existence of a connection, signal strength associated with the connection, response time data, and the like. When a location detection unit 129 detects the caregiver device 120 is proximate to a specific location, the location detection unit may generate and transmit a notification to the caregiver via the input/output interface 122. The notification may be in the form of, for example, a push notification, a text message, a voice message, and the like.

By way of example, caregiver device 120 may include a smart device, such as an iPhone, iPod, iPad, Apple Watch from Apple® or any other mobile device running Apple's iOS operating system, any device running Google's Android® operating system, including, for example, smartphones running the Android® operating system and other wearable mobile devices, such as Google Glass or Samsung Galaxy Gear Smartwatch, any device running Microsoft's Windows® Mobile operating system, and/or any other smartphone or like device.

As depicted in FIG. 1, system 100 may include data storage 130. Data storage 130 may be a stand-alone data storage and/or may be fully or partially incorporated in a caregiver device 120 and/or medical provider system 140. Data storage 130 may include population-based clinical data 132, medical ontology 134, patient-centered data 136, and/or prior iterations knowledge base 138.

Population-based clinical data 132 may include data from publically available clinical data such as patient records, vital sign data, diagnosis data, treated data, and/or the like. Population-based clinical data 132 may include data from, for example and by no means exclusive, MIMIC-III Critical Care and/or the Framingham Heart Study databases.

Medical ontology 134 may include, for example, a Medical Subject Headings (MeSH) knowledge base, Unified Medical Language System (UMS), and/or an Online Mendelian Inheritance in Man (OMIM) knowledge base. Patient-centered data 136 may include, for example, prior patient medical records, patient observation data (from the patient and/or a caregiver), personal emergency response system (PERS) data, wearable device data (e.g. heart rate, SpO2 levels, gait and/or movement data, and/or the like), medication adherence data, and/or prior/current medication data.

A prior iterations knowledge base 138 may include data retrieved and/or processed during a health proxy assistance session. A health proxy assistance session may include an interaction between a caregiver and a medical professional during a care recipient medical event. A prior iterations knowledge base 138 may be dynamically altered over time based on the queries to the various databases in data storage 130 such that the data stored in a prior iterations knowledge base 138 includes only information deemed relevant to a particular care recipient based on each health proxy assistance session. A prior iterations knowledge base may include cache data storage that may store versions of the prior iterations knowledge base during a health proxy assistance session for quick retrieval prior to being written into the prior iterations knowledge base 138.

System 100 may include a medical provider system 140 having, for example, an input/output interface 142, an operating system 144, a processor 146 including an image processor, and/or memory 148.

Input/output interface 142 may include for example, I/O devices, which may be configured to provide input and/or output to/from a medical provider system 140 (e.g., keyboard, mouse, touch input, camera, microphone, display, speakers, printers, modems, network cards, etc.). Input/output interface 142 also may include antennae, antennae interface(s) and/or network interface(s) that may provide or enable wireless and/or wire line digital and/or analog interface to one or more networks, such as network 110, over one or more network connections, a power source that provides an appropriate alternating current (AC) or direct current (DC) to power one or more components of medical provider system 140 and a bus that allows communication among the various components of medical provider system 140. Input/output interface 142 may include a display, which may include for example output devices, such as a printer, display screen (e.g., monitor, television, and the like), speakers, projector, and the like. Although not shown, medical provide system 140 may include one or more encoders and/or decoders, one or more interleavers, one or more circular buffers, one or more multiplexers and/or de-multiplexers, one or more permuters and/or depermuters, one or more encryption and/or decryption units, one or more modulation and/or demodulation units, one or more arithmetic logic units and/or their constituent parts, and the like.

Input/output interface 142 may be capable of utilizing standardized transmission protocols, for example but not by way of limitation, ISO/IEC 14443 A/B, ISO/IEC 18092, MiFare, FeliCa, tag/smartcard emulation, and the like. Also, input/output interface 142 may be able to utilize transmission protocols and methods that are developed in the future using other frequencies or modes of transmission. Input/output interface 142 may also be backwards-compatible with existing techniques, for example RFID. Also, input/output interface 142 may support transmission requirements to meet new and evolving standards including internet based transmission triggered by NFC.

Operating system 144 may store instructions that operate various hardware components of the medical provider system 140 such as input/output interface 142, processor/image processor 146, and/or memory 148. By way of example, operating system may store various setting parameters such as network setting parameters and/or input/output parameters. Applications, such as applications to process images and/or other medical data may have instructions stored in memory, such as memory 148.

Processor 146 may include for example, a data processor, an image processor, a natural language processor, and/or the like. Processor 146 may include hardware and/or software required to execute operating systems software and/or various health proxy assistance methods described herein.

Memory 148 may include various types of data storage. By way of example, memory 148 may include random access memory (RAM) and read only memory (ROM), which may be configured to access and store data and information and computer program instructions. Memory 148 may include cached data storage (e.g., CPU cache, GPU cache, digital signal processors, memory management unit, disk cache, web cache, and/or the like). Memory 148 may also include storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium), where the files that comprise an operating system, application programs including, for example, web browser application, email application and/or other applications, and data files may be stored. Memory 148 may include electronic information, files, and documents stored in various ways, including, for example, a flat file, indexed file, hierarchical database, relational database, such as a database created and maintained with software from, for example, Oracle® Corporation, Microsoft® Excel file, Microsoft® Access file, or any other storage mechanism.

By way of example, medical provider system may include a network-enabled computer and/or a smart device, such as an iPhone, iPod, iPad, Apple Watch from Apple® or any other mobile device running Apple's iOS operating system, any device running Google's Android® operating system, including, for example, smartphones running the Android® operating system and other wearable mobile devices, such as Google Glass or Samsung Galaxy Gear Smartwatch, any device running Microsoft's Windows® Mobile operating system, and/or any other smartphone or like device.

System 100 may further include a decision engine 150. Although shown as a stand-alone engine, decision engine 150 may be incorporated fully and/or in part into a caregiver device 120 and/or medical provider system 140. Decision engine 150 may include, for example, an input/output interface 152, an additional information processor 154, a natural language processor 156, and/or a machine learning processor 158.

Input/output interface 152 may include for example, I/O devices, which may be configured to provide input and/or output to/from decision engine 150 (e.g., keyboard, mouse, touch input, camera, microphone, display, speakers, printers, modems, network cards, etc.). Input/output interface 152 also may include antennae, antennae interface(s) and/or network interface(s) that may provide or enable wireless and/or wire line digital and/or analog interface to one or more networks, such as network 110, over one or more network connections, a power source that provides an appropriate alternating current (AC) or direct current (DC) to power one or more components of decision engine 150 and a bus that allows communication among the various components of decision engine 150. Input/output interface 152 may include a display, which may include for example output devices, such as a printer, display screen (e.g., monitor, television, and the like), speakers, projector, and the like. Although not shown, decision engine 150 may include one or more encoders and/or decoders, one or more interleavers, one or more circular buffers, one or more multiplexers and/or de-multiplexers, one or more permuters and/or depermuters, one or more encryption and/or decryption units, one or more modulation and/or demodulation units, one or more arithmetic logic units and/or their constituent parts, and the like.

Input/output interface 152 may be capable of utilizing standardized transmission protocols, for example but not by way of limitation, ISO/IEC 14443 A/B, ISO/IEC 18092, MiFare, FeliCa, tag/smartcard emulation, and the like. Also, input/output interface 152 may be able to utilize transmission protocols and methods that are developed in the future using other frequencies or modes of transmission. Input/output interface 152 may also be backwards-compatible with existing techniques, for example RFID. Also, input/output interface 152 may support transmission requirements to meet new and evolving standards including internet based transmission triggered by NFC.

Additional information processor 154 may work in combination with natural language processor 156 and/or machine learning processor 158 to determine if the decision engine needs additional information to determine a recommended treatment for the care recipient. Decision engine queries the various databases include data storage 130 and memory 126/memory 148 to determine a confidence associated with a recommended treatment and/or diagnosis.

By way of example, additional information processor 154 and/or machine learning processor 158 may determine a confidence interval associated with a diagnosis and/or treatment option. A confidence may be determined using, for example, statistical confidence, matched cases, and/or machine learning techniques such as decision trees, neural networks (e.g., artificial neural networks, ANN), deep learning, support vector machines (SVMs), clustering, Bayesian networks, and/or the like.

Additional information processor 154 and/or machine learning processor 158 may then determine that an additional data point (testing results, PERS event data, medication adherence data, and/or the like) that may increase a confidence interval associated with a diagnosis and/or treatment recommendation. Additional information processor 154 may then generate a question based on the additional data point which may then be transmitted to a caregiver via the caregiver device 120 to assist the caregiver in obtaining a best diagnosis and/or treatment recommendation for the care recipient. Should the caregiver not understand the question (determined via input received on the caregiver device 120, prior input received on the caregiver device 120, and/or other techniques), natural language processor 156 may translate the additional information request for the caregiver.

Upon receiving a response to the additional information request (either via the caregiver device 120 and/or medical provider system 140), a new confidence interval may be determined based on an updated data query and data processing. This process may repeat until a predetermined confidence interval threshold is reached.

Natural language processor 156 may also parse the received additional data (voice data associated with the medical professional, alphanumeric data associated with the medical professional, imagine data associated with the medial provider system 140 and/or data storage 130, and/or testing results associated with the medical provider system 140 and/or data storage 130.

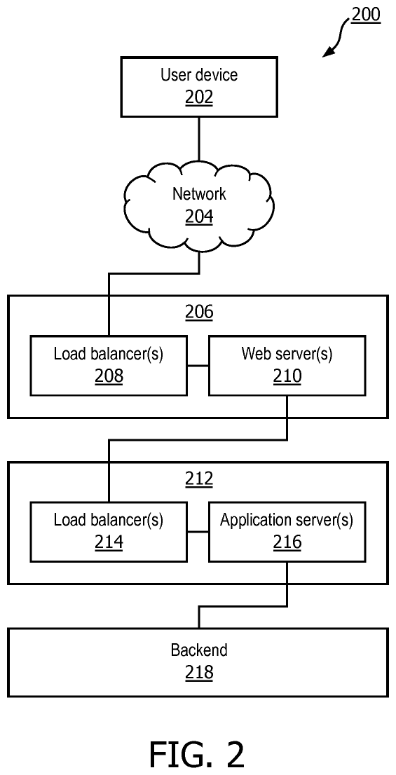

FIG. 2 depicts an example system that may be used in providing health proxy assistance sessions as described herein. The example system 200 in FIG. 2 may enable a medical provider system and/or decision engine, for example, to provide services to a caregiver, such as health proxy assistance sessions. As shown in FIG. 2, system 200 may include a user device 202, a network 204, a front-end controlled domain 206, a back-end controlled domain 212, and a backend 218. Front-end controlled domain 206 may include one or more load balancers 208 and one or more web servers 210. Back-end controlled domain 212 may include one or more load balancers 214 and one or more application servers 216.

User device 202, which may be a caregiver device such as caregiver device 120, may be a network-enabled computer. As referred to herein, a network-enabled computer may include, but is not limited to: e.g., any computer device, or communications device including, e.g., a server, a network appliance, a personal computer (PC), a workstation, a mobile device, a phone, a handheld PC, a personal digital assistant (PDA), a thin client, a fat client, an Internet browser, or other device. The one or more network-enabled computers of the example system 200 may execute one or more software applications to enable, for example, network communications.

User device 202 also may be a mobile device. For example, a mobile device may include an iPhone, iPod, iPad from Apple® or any other mobile device running Apple's iOS operating system, any device running Google's Android® operating system, including for example, Google's wearable device, Google Glass, any device running Microsoft's Windows® Mobile operating system, and/or any other smartphone or like wearable mobile device.

Network 204 may be one or more of a wireless network, a wired network, or any combination of a wireless network and a wired network. For example, network 204 may include one or more of a fiber optics network, a passive optical network, a cable network, an Internet network, a satellite network, a wireless LAN, a Global System for Mobile Communication (GSM), a Personal Communication Service (PCS), a Personal Area Networks, (PAN), D-AMPS, Wi-Fi, Fixed Wireless Data, IEEE 802.11b, 802.15.1, 802.11n, and 802.11g or any other wired or wireless network for transmitting and receiving a data signal.

In addition, network 204 may include, without limitation, telephone lines, fiber optics, IEEE Ethernet 902.3, a wide area network (WAN), a local area network (LAN) or a global network such as the Internet. Also, network 204 may support an Internet network, a wireless communication network, a cellular network, or the like, or any combination thereof. Network 204 may further include one network, or any number of example types of networks mentioned above, operating as a stand-alone network or in cooperation with each other. Network 204 may utilize one or more protocols of one or more network elements to which they are communicatively couples. Network 204 may translate to or from other protocols to one or more protocols of network devices. Although network 204 is depicted as a single network, it should be appreciated that according to one or more embodiments, network 204 may comprise a plurality of interconnected networks, such as, for example, the Internet, a service provider's network, a cable television network, corporate networks, and home networks.

Front-end controlled domain 206 may be implemented to provide security for backend 218. Load balancer(s) 208 may distribute workloads across multiple computing resources, such as, for example computers, a computer cluster, network links, central processing units or disk drives. In various embodiments, load balancer(s) 210 may distribute workloads across, for example, web server(S) 216 and/or backend 218 systems. Load balancing aims to optimize resource use, maximize throughput, minimize response time, and avoid overload of any one of the resources. Using multiple components with load balancing instead of a single component may increase reliability through redundancy. Load balancing is usually provided by dedicated software or hardware, such as a multilayer switch or a Domain Name System (DNS) server process.

Load balancer(s) 208 may include software that monitoring the port where external clients, such as, for example, cardholder device 202, connect to access various services of a financial institution, for example. Load balancer(s) 208 may forward requests to one of the application servers 216 and/or backend 218 servers, which may then reply to load balancer 208. This may allow load balancer(s) 208 to reply to cardholder device 202 without cardholder device 202 ever knowing about the internal separation of functions. It also may prevent cardholder devices from contacting backend servers directly, which may have security benefits by hiding the structure of the internal network and preventing attacks on backend 218 or unrelated services running on other ports, for example.

A variety of scheduling algorithms may be used by load balancer(s) 208 to determine which backend server to send a request to. Simple algorithms may include, for example, random choice or round robin. Load balancers 208 also may account for additional factors, such as a server's reported load, recent response times, up/down status (determined by a monitoring poll of some kind), number of active connections, geographic location, capabilities, or how much traffic it has recently been assigned.

Load balancers 208 may be implemented in hardware and/or software. Load balancer(s) 208 may implement numerous features, including, without limitation: asymmetric loading; Priority activation: SSL Offload and Acceleration; Distributed Denial of Service (DDoS) attack protection; HTTP/HTTPS compression; TCP offloading; TCP buffering; direct server return; health checking; HTTP/HTTPS caching; content filtering; HTTP/HTTPS security; priority queuing; rate shaping; content-aware switching; client authentication; programmatic traffic manipulation; firewall; intrusion prevention systems.

Web server(s) 210 may include hardware (e.g., one or more computers) and/or software (e.g., one or more applications) that deliver web content that can be accessed by, for example a client device (e.g., cardholder device 202) through a network (e.g., network 204), such as the Internet. In various examples, web servers, may deliver web pages, relating to, for example, health proxy assistance sessions, to clients (e.g., user device 202). Web server(s) 210 may use, for example, a hypertext transfer protocol (HTTP/HTTPS or sHTTP) to communicate with user device 202. The web pages delivered to user device may include, for example, HTML documents, which may include images, style sheets and scripts in addition to text content.

A user agent, such as, for example, a web browser, web crawler, or native mobile application, may initiate communication by making a request for a specific resource using HTTP/HTTPS and web server 210 may respond with the content of that resource or an error message if unable to do so. The resource may be, for example a file on stored on backend 218. Web server(s) 210 also may enable or facilitate receiving content from user device 202 so user device 202 may be able to, for example, submit network-based forms, including uploading of files.

Web server(s) also may support server-side scripting using, for example, Active Server Pages (ASP), PHP, or other scripting languages. Accordingly, the behavior of web server(s) 210 can be scripted in separate files, while the actual server software remains unchanged.

Load balancers 214 may be similar to load balancers 208 as described above.

Application server(s) 216 may include hardware and/or software that is dedicated to the efficient execution of procedures (e.g., programs, routines, scripts) for supporting its applied applications. Application server(s) 216 may comprise one or more application server frameworks, including, for example, Java application servers (e.g., Java platform, Enterprise Edition (Java EE), the .NET framework from Microsoft®, PHP application servers, and the like). The various application server frameworks may contain a comprehensive service layer model. Also, application server(s) 216 may act as a set of components accessible to, for example, a decision engine and/or medical facility system or other entity implementing system 200, through an API defined by the platform itself. For Web applications, these components may be performed in, for example, the same running environment as web server(s) 210, and application servers 216 may support the construction of dynamic pages. Application server(s) 216 also may implement services, such as, for example, clustering, fail-over, and load-balancing. In various embodiments, where application server(s) 216 are Java application servers, the web server(s) 216 may behaves like an extended virtual machine for running applications, transparently handling connections to databases associated with backend 218 on one side, and, connections to the Web client (e.g., client device 202) on the other.

Backend 218 may include hardware and/or software that enables the backend services of, for example, a decision engine and/or medical facility system or other entity that maintains a distributed system similar to system 200. For example, backend 218 may include, a system of one or more platforms that provide mobile services, one or more platforms that provide online services, a decision engine platform, and/or a location system, which may include additional capabilities, such as determining the location of a user device 202 and its proximity to a medical facility. Backend 218 may be associated with various databases, including data storage such as data storage 130. Backend 218 also may be associated with one or more servers that enable the various services provided by system 200.

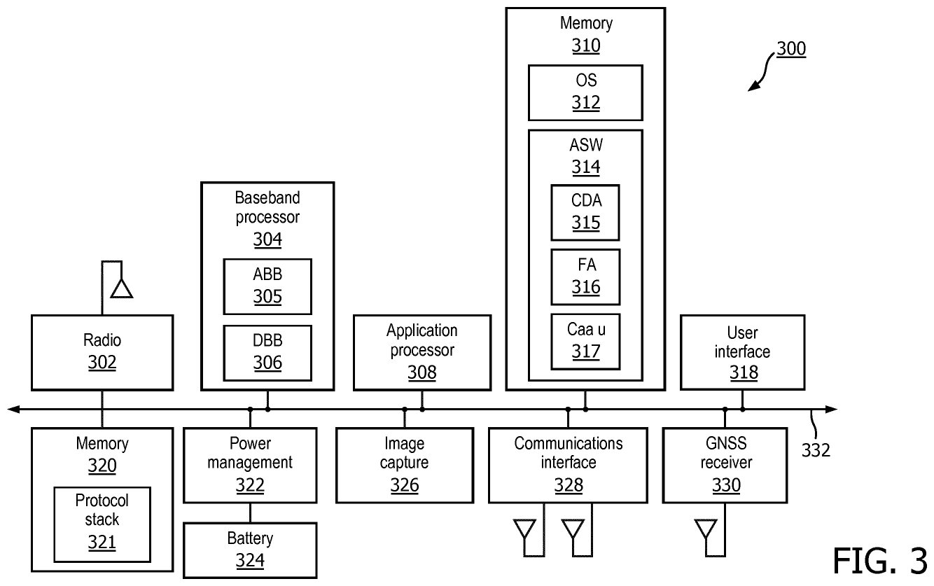

Referring now to FIG. 3, shown is an example mobile device 300 in which all or a portion of the functionality of a caregiver device 120 and/or medical facility system 140 may be implemented. In particular, FIG. 3 illustrates an example architecture (e.g., hardware and software) for the example mobile device 300. It should be appreciated by one having ordinary skill in the art in the context of the present disclosure that the architecture of the mobile device 300 depicted in FIG. 3 is but one example, and that in some embodiments, additional, fewer, and/or different components may be used to achieve similar and/or additional functionality. In the depicted example, the mobile device 300 is embodied as a smartphone, though in some embodiments, other types of devices may be used, including a workstation, laptop, notebook, tablet, etc. The mobile device 300 may be used in some embodiments to provide the entire functionality of certain embodiments of a health proxy assistance system 100, or in some embodiments, provide functionality of the caregiver device 120 in conjunction with one or any combination of data storage 130, medical facility system and/or decision engine 140.

The mobile device 300 comprises at least two different processors, including a baseband processor (BBP) 304 and an application processor (APP) 308. As is known, the baseband processor 304 primarily handles baseband communication-related tasks and the application processor 308 generally handles inputs and outputs and all applications other than those directly related to baseband processing. The baseband processor 304 comprises a dedicated processor for deploying functionality associated with a protocol stack, such as but not limited to a GSM (Global System for Mobile communications) protocol stack, among other functions. The application processor 308 comprises a multi-core processor for running applications, including all or a portion of application software. The baseband processor 304 and the application processor 308 have respective associated memory including random access memory (RAM), Flash memory, etc., and peripherals, and a running clock. The memory are each also referred to herein as a non-transitory computer readable medium. Note that, though depicted as residing in memory 310, all or a portion of the application software may be stored in memory 310, distributed among memory or reside in other memory.

The baseband processor 304 may deploy functionality of the protocol stack to enable the mobile device 300 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 304 manages radio communications and control functions, including signal modulation, radio frequency shifting, and encoding. The baseband processor 304 comprises, or may be coupled to, a radio (e.g., RF front end) 302 and/or a GSM (or other communications standard) modem, and analog and digital baseband circuitry (ABB, DBB, receptively). The radio 302 comprises one or more antennas, a transceiver, and a power amplifier to enable the receiving and transmitting of signals of a plurality of different frequencies, enabling access to a cellular (and/or wireless) network. The analog baseband circuitry is coupled to the radio 302 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 308 or directly from a user interface (UI) 318 (e.g., microphone, earpiece, ring tone, vibrator circuits, touch-screen, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 308). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 308 operates under control of an operating system (OS) that enables the implementation of a plurality of user applications, including, for example, the application software that supports a health proxy assistance session. The application processor 308 may be embodied as a System on a Chip (SOC), and supports a plurality of multimedia related features including web browsing/cloud-based access functionality to access one or more computing devices, of the cloud(s), that are coupled to the Internet. For instance, the application processor 308 may execute communications functionality of the application software (e.g., middleware, similar to some embodiments of the caregiver device 120 which may include a browser with or operable in association with one or more application program interfaces (APIs)) to enable access to a cloud computing framework or other networks to provide remote data access/storage/processing, and through cooperation with an embedded operating system, access to calendars, location services, user data, public data, caregiver observation data, care recipient medical data, etc.

For instance, in some embodiments, the health proxy assistance system may operate using cloud computing services, where the processing of raw and/or derived parameter data received, indirectly via the mobile device 300 or directly from the caregiver device 120, data storage 130, and/or medical provider system 140 may be achieved by one or more devices of the cloud(s), and triggering signals (to trigger feedback) may be communicated from the cloud(s) (or other devices) to the mobile device 300, which in turn may activate feedback internal to the mobile device 300 (e.g., visually, audibly, or via tactile mechanisms) or relay the triggering signals to other devices (e.g., medical provider system 140, decision engine 150).

The application processor 308 generally comprises a processor core (Advanced RISC Machine or ARM), and further comprises or may be coupled to multimedia modules (for decoding/encoding pictures, video, and/or audio), a graphics processing unit (GPU), communications interface 328, and device interfaces. In one embodiment, the communications interfaces 328 may include wireless interfaces, including a Bluetooth (BT) (and/or Zigbee in some embodiments, among others) module that enable wireless communication with the data storage 130, medical provider system 140, and/or decision engine 140.

In some embodiments, the communications interface 328 may comprise a Wi-Fi module for interfacing with a local 802.11 network, according to corresponding communications software in the applications software. The application processor 308 further comprises, or in the depicted embodiment, is coupled to, a global navigation satellite systems (GNSS) receiver 330 for enabling access to a satellite network to, for instance, provide position coordinates. In some embodiments, the GNSS receiver 330, in association with GNSS functionality in the application software, collects contextual data (time and location data, including location coordinates and altitude) to determine a proximity to a medical facility for example. Note that, though described as a GNSS receiver 330, other indoor/outdoor positioning systems may be used, including those based on triangulation of cellular network signals and/or Wi-Fi.

The device interfaces coupled to the application processor 308 may include the user interface 318, including a display screen. The display screen, in some embodiments similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), retina or haptic-based technology, or virtual/augmented reality technology. For instance, the user interface 318 may present visual feedback in the form of messaging (e.g., text messages) and/or symbols/graphics (e.g., warning or alert icons, flashing screen, etc.), and/or flashing lights (LEDs). In some embodiments, the user interface 318 may be configured, in addition to or in lieu of a display screen, a keypad, microphone, speaker, ear piece connector, I/O interfaces (e.g., USB (Universal Serial Bus)), SD/MMC card, among other peripherals. For instance, the speaker may be used to audibly provide feedback, and/or the user interface 318 may comprise a vibratory motor that provides a vibrating feedback to the user. One or any combination of visual, audible, or tactile feedback may be used, and as described before, variations in the intensity of format of the feedback may be used.

Also coupled to the application processor 308 is an image capture device (IMAGE CAPTURE) 326. The image capture device 326 may comprise an optical sensor (e.g., a charged coupled device (CCD) or a complementary metal-oxide semiconductor (CMOS) optical sensor). The image capture device 326 may be configured to capture and/or receive medical imaging, such as ultrasounds and/or other scans.

Also included is a power management device 322 that controls and manages operations of a battery 324. The components described above and/or depicted in FIG. 3 share data over one or more busses, and in the depicted example, via data bus 332. It should be appreciated by one having ordinary skill in the art, in the context of the present disclosure, that variations to the above may be deployed in some embodiments to achieve similar functionality.

In the depicted embodiment, the application processor 308 runs the application software 314, and may comprise a conversation detection application 315, a feedback application 316, and a communications application 317. The conversation detection application 315 may, upon receiving an indication to begin a healthy proxy assistance session, execute voice recognition processes to detect a caregiver voice and a medical professional (or other) voice. Conversation detection application 315 may use natural language processing techniques to parse the language spoked by the medical professional to determine any medical data (testing results, vital signs, emergency medication provision, and/or the like) associated with a care recipient medical event. This data may then be used by a decision engine to successfully execute a health proxy assistance session. Conversation detection application 315 may also receive input via an interface in the form of alphanumeric input, imaging input, and/or the like to detect and determine relevant medical data for a health proxy assistance session.

The feedback application provides for visual, audible, and or tactile feedback to the user via the user interface 318. The communications interface 328 communicates raw and/or derived parameters to one or more other devices such as data storage 130, medical provider system 140, and/or decision engine 150, and also receives triggering signals to activate the feedback functionality. For instance, in one embodiment, the mobile device 300 communicates parameters to the data storage 130 and/or decision engine 150 and receives feedback from these system components.

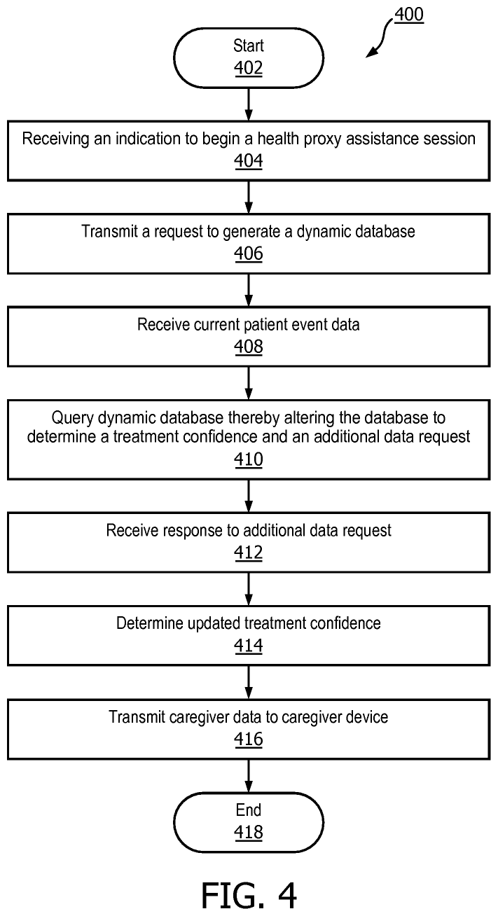

FIG. 4 depicts an exemplary method 400 employed by the system components described herein to provide a health proxy assistance session. Method 400 may begin at 402.

At block 404, a system may receive an indication to begin a medical guidance or health proxy assistance session. The indication may be received in response to manual input via an input/output interface on a caregiver device. The indication may be receive in response to a prompt and/or push notification generated upon the caregiver device detecting a proximity to a medical facility. Caregiver device may detect proximity to a medical facility by determining that a caregiver device is within a predefined distance of a medical facility using location detection technology and/or proximity data received from a medical facility device (e.g., Bluetooth, BLE, RFID WiFi and/or other network data indicating network data of a medical facility). The indication may be received automatically upon a caregiver device detecting a proximity to a medical facility.

Upon receiving an indication to begin a health proxy assistance session, a security setting may be determined associated with the caregiver device. A security setting may be based on network settings associated with the network connected to the caregiver device. An indication to begin a health proxy assistance session may include transmitting a security feature based on the security setting comprising at least one of: a username, a password, a personal identification number (PIN), and a biometric data. Moreover, prior to and/or in conjunction with receiving the indication to begin the medical guidance session, a caregiver device may receive a notification to change a network setting to increase security. By way of example, a caregiver device may receive a notification to leave a wide area network and join a local area network. Caregiver device may automatically resume prior network settings once a health proxy assistance session ends.

At block 406, a caregiver device may transmit a request to generate a dynamic database. For example, a caregiver device, such as caregiver device 120 may query data storage such as data storage 130 to determine care recipient-specific data as well as medical event specific data and/or population-specific data. For example, where a care recipient has suffered a particular medical event, data associated with that particular event (agnostic to the care recipient) may be retrieved and/or population-based clinical data, such as data specific to demographically-similar and/or medically-similar patients may be retrieved to generate a dynamic database for a particular health proxy assistance session. Dynamic database may further include ontology data, such as data relevant to the current care recipient medical event from a Medical Subject Headings (MeSH) knowledge base, Unified Medial Language System (UMLS), and/or the Online Mendelian Inheritance in Man (OMIM) knowledge bases. Dynamic database may further include patient-centric data relevant to the current care recipient medical event such as prior medical records, caregiver observation data, PERS data, care recipient wearable device data (fall detection data, vital signs data, stress data, activity data, and/or the like), medication adherence data, and/or current or prior medication data. Dynamic database may also include patient specific data such as patient insurance data.

Once relevant data is retrieved from population-based clinical data, ontology data, and patient-centric data, the data may be combined into a dynamic database that is cached in memory, for example of the decision engine, during the healthy proxy assistance session until it is written into memory (of a prior iterations database, caregiver device memory, medical provider system memory and/or decision engine).

While a dynamic database is being generate, a caregiver device may receive current patient event data at block 408 from a medical professional and/or a medical provider system. Current patient event data may include voice data from a medical professional detailing the medical event, testing and/or examination results, treatment recommendation(s), and/or diagnosis. Current patient event data may include medical testing and/or imaging results. Current patient event data may include real-time vital signs measurements. A dynamic database may be continuously generated upon receipt of each data point associate with the current patient event data.

At block 410, the dynamic database may be queried to determine a confidence interval associated with a diagnosis and/or treatment recommendation using, for example, statistical confidence, matched cases, and/or machine learning techniques such as decision trees, neural networks (e.g., artificial neural networks, ANN), deep learning, support vector machines (SVMs), clustering, Bayesian networks, and/or the like. In this way, a diagnosis and/or treatment recommendation may be associated with a confidence interval. Decision engine may then determine additional data that may increase the confidence level associated with a particular diagnosis and/or treatment option by altering the variables of the matched cases, determining which variable changes may increase the confidence level, and selecting the variable that make the most impact on a confidence level. An additional data request may be generated using natural language processing to generate a caregiver-level question and/or request for information to be answered by the medical professional. A caregiver-level may be determined to include medical terminology as well as a descriptive terminology that explains the medical terminology. By way of example, a natural language processing technique may be used to replace medical terminology, such as transient ischemic attack, with more descriptive terminology, such as deprive blood flow to the brain. In this manner, the caregiver may be able to ask questions and receive information that the caregiver is able to comprehend.

By way of example, a diagnosis of Congestive Heart Failure (CHF) may be originally provided and a treatment recommendation may be fluid removal. Upon querying the dynamic database it may be determined that pulmonary consult test result data is a variable that would most alter a confidence of the diagnosis and/or treatment recommendation. Accordingly, the decision engine may generate a question to determine if the treatment may be altered to include a pulmonary consult test. Upon receiving test results, the decision engine may query the dynamic database with the updated information and determine a lower confidence of the original diagnosis and treatment and a higher confidence of an alternate diagnosis (e.g., pulmonary embolism) and treatment (e.g., blood thinning treatment). The natural language processor may explain what a pulmonary consult test is and generate a question to ask if the patient can receive that test.

At block 412, a response may be received to the additional data request. In the example above, the response may be a doctor explaining the results of the test to the caregiver and/or the actual result data itself. Additional data request may include data relevant to the care recipient or patient's insurance. In block 414, an updated treatment confidence may be determined. In the example above, it may be determined that the treatment confidence for CFH is lower and the treatment confidence for a pulmonary embolism is higher.

At block 416, caregiver data may be transmitted to the caregiver via the caregiver device. For example, caregiver data may include an explanation of the diagnosis, an explanation of the treatment recommendation, a treatment and/or diagnosis confidence, and/or links for additional data regarding the diagnosis and/or treatment.

At block 418, the method may end.

It is further noted that the systems and methods described herein may be tangibly embodied in one of more physical media, such as, but not limited to, a compact disc (CD), a digital versatile disc (DVD), a floppy disk, a hard drive, read only memory (ROM), random access memory (RAM), as well as other physical media capable of storing software, or combinations thereof. Moreover, the figures illustrate various components (e.g., servers, computers, processors, etc.) separately. The functions described as being performed at various components may be performed at other components, and the various components bay be combined or separated. Other modifications also may be made.

The present disclosure is not to be limited in terms of the particular embodiments described in this application, which are intended as illustrations of various aspects. Many modifications and variations can be made without departing from its spirit and scope, as may be apparent. Functionally equivalent methods and apparatuses within the scope of the disclosure, in addition to those enumerated herein, may be apparent from the foregoing representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It may be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It may be further understood by those within the art that if a specific number of an introduced claim recitation is intended, such an intent may be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It may be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” may be understood to include the possibilities of “A” or “B” or “A and B.”

The foregoing description, along with its associated embodiments, has been presented for purposes of illustration only. It is not exhaustive and does not limit the invention to the precise form disclosed. Those skilled in the art may appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the invention is not limited to the above-described embodiments, but instead is defined by the appended claims in light of their full scope of equivalents.

In the preceding specification, various preferred embodiments have been described with references to the accompanying drawings. It may, however, be evident that various modifications and changes may be made thereto, and additional embodiments may be implemented, without departing from the broader scope of the invention as set forth in the claims that follow. The specification and drawings are accordingly to be regarded as an illustrative rather than restrictive sense.

1. A device comprising:
an antenna that communicates wirelessly with a network;
an input/output interface comprising a display; and
a processor that:
receives an indication to begin a medical guidance session;
transmits, via the antenna and in response to receiving an indication to begin a medical guidance session, a request to generate a dynamic database comprising patient specific data, population-based clinical data, and medical ontology data, wherein the dynamic database is dynamically reduced in response to each query to the dynamic database during the medical guidance session;
receives current patient event data comprising a treatment recommendation and at least one of: diagnosis data, clinical notes, and image data;
executes a query process comprising:
querying the dynamic database using the current patient event data to identify a refined dataset used to determine (a) a preliminary treatment confidence associated with the treatment recommendation and (b) additional data that could increase the preliminary treatment confidence;
generating a question for the user, wherein the question is associated with the determined additional data;
receiving a response to the generated question via the input/output interface; and
determining an updated treatment confidence associated with the treatment recommendation; and
transmits, via the input/output interface, caregiver data comprising at least one of: the updated treatment confidence and a description of the treatment recommendation associated with the medical guidance session.

2. The device of claim 1, wherein the processor further determines a security setting based on network settings associated with the network, and wherein the indication to begin a medical guidance session comprises a security feature based on the security setting comprising at least one of: a username, a password, a personal identification number (PIN), and a biometric data.

3. The device of claim 1, wherein the processor, prior to receiving the indication to begin the medical guidance session, transmits a notification to change a network setting to increase security.

4. The device of claim 1, wherein the patient specific data comprises at least one of: prior medical record data, caregiver observation data, personal emergency response system (PERS) data, care recipient wearable device data, medication adherence data, medication data and patient insurance data, and
further wherein when the patient specific data comprises patient insurance data, at least one of the preliminary treatment confidence and updated treatment confidence is based on the patient insurance data, and the generated question is related to the patient insurance data.

5. The device of claim 1, wherein the query process is repeated until the updated treatment confidence is above a predetermined threshold, wherein for each repetition, the preliminary treatment confidence is substituted with a further updated treatment confidence.

6. The device of claim 1, wherein the treatment recommendation is changed when the updated treatment confidence associated with the treatment recommendation drops below a predetermined threshold.

7. (canceled)

8. The device of claim 1 further comprising:
a location detection unit that detects a location of the device, wherein when the location of the device is within a predefined distance of a medical facility, the processor generates a notification to begin the medical guidance session prior to receiving an indication to begin the medical guidance session,
optionally wherein when the location detection unit determines the location of the device is within a predefined distance of a medical facility, the device generates a notification to alter a network setting, wherein the notification comprises selectable data to automatically alter the network setting.

9. (canceled)

10. The device of claim 1 further comprising a camera and/or memory, wherein if the device comprises a camera, image data is received via the camera, and wherein if the device comprises a memory, the dynamic database is cached in the memory.

11. The device of claim 1, wherein the treatment recommendation comprises at least two treatment options, and wherein the preliminary treatment confidence comprises a treatment option confidence for each treatment option, and wherein the determined additional data could increase the treatment option confidence for at least one of the treatment options.

12. (canceled)

13. The device of claim 1, wherein the input/output interface further comprises a microphone, and wherein at least a portion of the current patient event data is received via the microphone.

14. The device of claim 1, wherein the current patient event data is preprocessed using natural language processing.

15. The device of claim 1, wherein the caregiver data is preprocessed prior to transmission, wherein the preprocessing of the caregiver data comprises performing natural language processing on at least one of the updated treatment confidence and the description of the treatment recommendation.

16. The device of claim 1, wherein determining the preliminary treatment confidence and the updated treatment confidence comprises using a machine learning technique comprising at least one of: decision tree learning, association rule learning, neural networks, deep learning, inductive logic programming (ILP), a support vector machine (SVM), clustering, Bayesian networks, reinforcement learning, and a learning classifier system (LCS),
optionally wherein the preliminary treatment confidence and the updated treatment confidence are each based on matched cases.

17. The device of claim 1, wherein the caregiver data further comprises at least one follow-up question.

18. (canceled)

19. The device of claim 1, wherein the antenna is configured such that it becomes inactive once the dynamic database is cached on the device and/or such that it remains inactive until the medical guidance session ends.

20. (canceled)
